(19) United States
(12) Patent Application Publication
(10) Pub. No.: US 2026/0165961 A1
(43) Pub. Date: Jun. 18, 2026

(54) APPLICATION AID FOR AN ORAL THIN FILM

(71) Applicant: LTS Lohmann Therapie-Systeme AG, Andernach (DE)

(72) Inventor: Markus MÜLLER, Troisdorf (DE)

(21) Appl. No.: 18/713,431

(22) PCT Filed: Nov. 25, 2022
(86) PCT No.: PCT/EP2022/083375

§ 371 Date: May 24, 2024

(30) Foreign Application Priority Data
Nov. 25, 2021 (DE) 10 2021 130 950.6

(51) Int. Cl. A61K 9/00; A61K 9/70; A61M 37/00

(52) U.S. Cl. CPC A61K 9/006; A61K 9/7007; A61M 37/0015; A61M 2037/0023

(57) ABSTRACT

The invention relates to a composite comprising an oral thin film and an application aid for said oral thin film, wherein the oral thin film and the application aid are firmly joined to each other, a method for its preparation, and the composite for use as a medicinal product.

The invention relates to a composite comprising an oral thin film and an application aid for said oral thin film, a method for its preparation and the composite for use as a medicinal product.

Oral thin films are thin films, usually containing an active pharmaceutical ingredient, which are placed directly in the oral cavity or applied to the oral mucosa, where they dissolve. In particular, they are thin polymer-based films containing active ingredients which, when applied to a mucous membrane, especially the oral mucosa, release the active ingredient directly into it. These oral thin films are generally not tacky on the outside. The excellent blood circulation of the oral mucosa ensures that the active ingredient is quickly absorbed into the bloodstream. This dosage system has the advantage that the active ingredient is largely absorbed through the mucous membrane, thus avoiding the “first-pass metabolism” that occurs with the conventional dosage form of an active ingredient in tablet form, which is usually taken with liquid, which can be disadvantageous. The active ingredient can be dissolved, emulsified, or dispersed in the film. Suitable active ingredients can also be swallowed after dissolving the oral thin film in the mouth and thus be absorbed via the gastrointestinal tract.

The application of oral thin films can be problematic. Due to the shape, size, properties, and intended application site of the oral thin film, but also due to the user's impairments, difficulties may arise in applying the oral thin film to the desired site. Particularly in the case of oral thin films, which are equipped with microneedles on one surface to increase the active ingredient flux, it is important that these microneedles are not damaged before any application.

Conventional application aids can improve this, but they usually have to be laboriously removed from the oral thin film and/or have a complicated structure. There is also a risk that the user may inadvertently swallow the application aid.

The present application therefore was based on the problem of providing an application aid for an oral thin film which overcomes the above disadvantages. The application aid should have a high haptic stability in order to apply the oral thin film at the desired location. In addition, the risk of swallowing the application aid as a whole should be minimized, and the risk of the user destroying or damaging the oral thin film before application should be minimized.

This task was surprisingly solved with a composite according to claim 1, in particular with a composite comprising an oral thin film and an application aid for the oral thin film, wherein the oral thin film and the application aid are firmly joined to each other.

The advantage of such a joint is that the user does not have to touch the oral thin film itself in order to apply it. The contact is preferably made exclusively via the application aid. Furthermore, in a preferred embodiment, the application aid can be designed in such a way that it dissolves in the oral cavity of the user. Due to the firm joint with the application aid, very thin oral thin films, in particular those with a thickness of less than 400 μm, or oral thin films that are equipped with microneedles on one surface, can be applied advantageously.

In the present description, the term “comprise” can also mean “consisting of”.

The term “application aid” can be understood in the broadest possible sense here.

Preferably, the application aid comprises a flat structure with a preferred thickness of 0.1 mm to 4 mm, preferably from 0.1 mm to 2 mm, or 0.5 mm to 2 mm, or from 100 μm to 500 μm, to the surface of which the oral thin film is firmly attached.

The application aid can take any form here. For example, the application aid can be essentially round, oval, or elliptical.

However, the application aid can also be triangular or square, if necessary with rounded corners. Shapes in other possible polygons, if necessary with rounded corners, are also possible.

Mixed forms are possible as well, wherein the application aid has, for example, a first round, oval, or elliptical area and a second area attached to it, which is, for example, strip-shaped, triangular, or square (also polygonal), so that the application aid as a whole has a shape that resembles the shape of a tennis racket, wherein the second area here preferably serves as a “handle” and the first area serves to hold the oral thin film.

In the present case, “firmly joined” means that the application aid and the oral thin film are joined to each other in such a way that the user cannot separate the joint without destroying the film. This joint should also not be impaired over a longer storage period.

The composite according to the invention is preferably characterized in that the application aid comprises a first area and a second area.

The oral thin film is preferably disposed in the first area of the application aid.

The second area of the application aid is preferably designed in such a way that a user can touch the application aid in such a way that the oral thin film is not touched.

Preferably, the oral thin film is attached to a first third or a first half of the surface of the application aid. This leaves two thirds or a second half of the application aid free, which can serve as a “handle” for applying the oral thin film.

The composite according to the invention is preferably characterized in that the application aid has a surface area which is at least twice as large, preferably 2 to times or 2 to 4 times as large, as the surface area of the oral thin film.

In possible embodiments, the application aid has a surface area of 1 cm² to 30 cm², preferably from 2 cm² to 15 cm², or 2 cm² to 10 cm².

In possible embodiments, the oral thin film has a surface area of 0.3 cm² to 10 cm², preferably 0.5 cm² to 7 cm².

In possible embodiments, the application aid has a length of 1 cm to 10 cm, preferably 2 cm to 5 cm.

In possible embodiments, the application aid has a width of 0.5 cm to 5 cm, preferably from 1 cm to 3 cm.

In possible embodiments, the oral thin film has a length of 0.5 cm to 5 cm, preferably from 1 cm to 4 cm.

In possible embodiments, the oral thin film has a width of 0.5 cm to 4 cm, preferably from 1 cm to 4 cm.

In possible embodiments, the application aid has a thickness of 0.1 mm to 4 mm, preferably 0.1 mm to 2 mm, or 100 μm to 500 μm.

In possible embodiments, the oral thin film has a thickness of 0.01 mm to 2 mm, preferably 0.05 mm to 1 mm, or 100 μm to 400 μm.

In possible embodiments, the application aid has a weight per unit area of 20 g/m² to 800 g/m², more particularly 20 g/m² to 500 g/m².

In possible embodiments, the oral thin film has a weight per unit area of 10 g/m² to 600 g/m², preferably 10 g/m² 400 g/m².

The application aid can be formed single-layered or multi-layered.

The composite according to the invention is preferably characterized in that the application aid comprises at least one water-soluble polymer.

Water-soluble polymers comprise natural or synthetic polymers that are chemically quite different and whose common characteristic is their solubility in water or aqueous media. A prerequisite is that these polymers have a sufficient number of hydrophilic groups for water solubility and are not cross-linked. The hydrophilic groups may be non-ionic, anionic, cationic, and/or zwitterionic.

Water-soluble is preferably understood to mean a solubility of more than 100 g/L in water at 25° C.

The at least one water-soluble polymer is preferably selected from the group consisting of starch and starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acids, polyacrylates, polyvinylpyrrolidones, vinylpyrrolidone-vinyl acetate copolymers, polyvinyl alcohols, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan, and/or natural gums, polyvinyl alcohols being particularly preferred.

The composite according to the invention is preferably characterized in that the at least one polymer, preferably the water-soluble polymer, is present in the matrix layer in an amount of 10 to 100% (w/w), or 10 to 95% (w/w), preferably 60 to 90% (w/w), particularly preferably 80 to 90% (w/w), based on the total weight of the application aid.

The composite according to the invention is preferably characterized in that the application aid as a whole is water-soluble. Here too, water-soluble is preferably understood to mean a solubility of more than 100 g/L in water at 25° C.

The composite according to the invention is preferably characterized in that the application aid comprises aromas, taste masking agents, and/or anesthetics or local anesthetics.

As anesthetics or local anesthetics, aminoamides such as lidocaine, amino esters such as procaine, fomocaine, quinisocaine, or cyclonine are suitable here.

These are preferably contained in an amount of 0.1 to 10% (w/w) or 0.1 to 5% (w/w), based on the total weight of the application aid.

The composite according to the invention is preferably characterized in that the application aid further comprises at least one excipient selected from the group comprising colorants, sweeteners, plasticizers, emulsifiers, enhancers, pH regulators, humectants, preservatives, and/or antioxidants.

Each of these excipients is preferably present in an amount of 0.1 to 40% (w/w), respectively, preferably 0.1 to 30% (w/w), particularly preferably 0.1 to 15% (w/w), most preferably 0.1 to 10% (w/w), or 0.1 to 5% (w/w), based on the total weight of the application aid.

In particular, the stability of the application aid can be adjusted via the content of plasticizer.

Suitable plasticizers comprise glycerol.

The plasticizer(s) is/are preferably present in an amount of 0.1 to 15% (w/w), most preferably 0.1 to 10% (w/w), or 0.1 to 5% (w/w), particularly about 5% (w/w), based on the total weight of the application aid.

Preferably, the composite according to the invention is characterized in that the application aid is in the form of a film that is as smooth as possible.

In another embodiment, the composite according to the invention is characterized in that the application aid is in the form of a solidified foam having cavities.

The cavities and the associated larger surface area of the application aid particularly facilitate the access of water or saliva or other body fluids into the interior of the application aid, thus accelerating the dissolution of the application aid.

On the other hand, the wall thickness of the cavities mentioned is preferably low, as these represent solidified bubbles, for example, so that these cavities dissolve or are destroyed quickly.

A further advantage of this embodiment is that, despite the comparatively high weight per unit area due to the preparation as a foam, a faster drying time can be achieved than with a comparable non-foamed composition.

Preferably, the composite according to the invention is characterized in that the cavities are isolated from each other and are preferably in the form of bubbles, wherein the cavities are filled with air or a gas, preferably with an inert gas, particularly preferably with nitrogen, carbon dioxide, helium, or a mixture of at least two of these gases.

According to another embodiment, it is provided that the cavities are connected with each other, preferably by forming a continuous channel system penetrating the matrix.

Preferably, the aforementioned cavities have a volume fraction of 5 to 98%, preferably 50 to 80%, based on the total volume of the application aid. In this way, the solution of the application aid is influenced in a favorable way.

Furthermore, surface-active substances or surfactants can be added to the application aids for foam formation or to the resulting foam before or after drying in order to improve the stability of the foam before or after drying.

Another parameter that influences the properties of the application aid according to the invention is the diameter of the cavities or bubbles. The bubbles or cavities are preferably produced with the aid of a foam whipping machine, with which the diameter of the bubbles can be adjusted over a wide range, almost at will. Thus, the diameter of the bubbles or cavities can range from 0.01 to 350 μm. Particularly preferably, the diameter is in the range of 10 and 200 μm.

Preferably, the composite according to invention is characterized in that the oral thin film comprises an oral thin film in the form of a film, in the form of a foam, and/or in the form of a microneedle system.

The oral thin film in the composite according to the invention preferably comprises a matrix layer.

A film is preferably characterized by the fact that it is not in the form of a foam and does not exhibit any significant unevenness when analyzed optically without aids or magnification. A film can be produced, for example, by spreading and drying a polymer-containing solution, emulsion, suspension, or melt.

In another embodiment, the oral thin film is characterized in that the matrix layer or the oral thin film is present in the form of a solidified foam having cavities.

The cavities and the associated larger surface area of the films facilitate the access of water or saliva or other body fluids into the interior of the dosage form, thus accelerating the dissolution of the dosage form and the release of the active ingredient.

In the case of a rapidly absorbing active ingredient, transmucosal absorption can also be improved due to the rapid dissolution.

On the other hand, the wall thickness of the cavities mentioned is preferably low, as these represent solidified bubbles, for example, so that these cavities dissolve or are destroyed quickly.

A further advantage of this embodiment is that, despite the comparatively high weight per unit area, a faster drying time can be achieved than with a comparable non-foamed composition due to the preparation as a foam.

Preferably, the oral thin film is characterized in that the cavities are isolated from each other and are preferably in the form of bubbles, wherein the cavities are filled with air or a gas, preferably with an inert gas, particularly preferably with nitrogen, carbon dioxide, helium, or a mixture of at least two of these gases.

According to another embodiment, it is provided that the cavities are connected with each other, preferably by forming a continuous channel system penetrating the matrix.

Preferably, the aforementioned cavities have a volume fraction of 5 to 98%, preferably 50 to 80%, based on the total volume of the matrix layer. In this way, the advantageous effect of accelerating the dissolution of the matrix layer is favorably influenced.

Furthermore, surface-active substances or surfactants can be added to the matrix for foam formation or to the resulting foam before or after drying in order to improve the stability of the foam before or after drying.

Another parameter that influences the properties of the dosage form according to the invention is the diameter of the cavities or bubbles. The bubbles or cavities are preferably produced with the aid of a foam whipping machine, with which the diameter of the bubbles can be adjusted over a wide range, almost at will. Thus, the diameter of the bubbles or cavities can range from 0.01 to 350 μm. Particularly preferably, the diameter is in the range of 10 and 200 μm.

A microneedle system, also known as a microneedle array, preferably comprises a system comprising a plurality of microneedles on a support.

The carrier preferably comprises an oral thin film based on at least one polymer. The needles, which preferably have a length of 5 μm to 1000 μm, can be made of different materials such as ceramics, steel, polymers or SiO₂.

The microneedle system preferably comprises microneedles with a length of 100 μm to 600 μm, preferably of 250 μm to 350 μm, particularly preferably of about 300 μm.

The composite according to the invention is preferably characterized in that the microneedle system is preferably a microneedle system based on glass, SiO₂, steel, ceramics, starch and starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acids, polyacrylates, polyvinylpyrrolidones, polyvinyl alcohols, poly(lactide-co-glycolide), hyaluronic acid, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan, and natural gums, wherein this group is not exhaustive.

The microneedles are preferably attached to the oral thin film with an adhesive layer. For example, an adhesive layer such as that defined for connecting the application aid to the oral thin film is suitable as an adhesive layer.

Alternatively, the oral thin film with microneedles can also be produced by using a negative mold of the microneedles during the production of the oral thin film, so that the oral thin film and the microneedles are monolithic.

Alternatively, the microneedles can be attached to the oral thin film using any suitable means of attachment.

Preferably, the needles comprise a polymer that is also present in the support.

The composite according to the invention is preferably characterized in that, if a microneedle system is present, in particular adjacent to the microneedles, there is a raised area which is preferably attached to the application aid.

The raised area is preferably designed in such a way that it protects the microneedles, in particular because the raised area is formed higher than the microneedles, so that the raised area is touched before the microneedles are.

The raised area is further preferably characterized in that the enclosed area comprises at least the same area as the microneedle system, particularly preferably the area of the oral thin film, and the height of the raised area corresponds at least to the height of the microneedles.

This ensures that the microneedles are protected from all sides by the raised area.

Preferably, said raised area is shaped in the form of a continuous molded body and frames a square, rectangular, elliptical, or circular base surface, wherein this is particularly adapted to the arrangement of the microneedle system.

In another embodiment, the raised area is not a continuous molded body, but partially frames the microneedle system.

The raised area is preferably characterized in that the raised area comprises a water-soluble polymer (as defined above).

Preferably, the raised area comprises the same material as the application aid.

Regardless of the form of the oral thin film, the oral thin film is preferably characterized in that the oral thin film comprises at least one water-soluble polymer (as defined above).

The at least one water-soluble polymer is preferably selected from the group consisting of starch and starch derivatives, dextrans, cellulose derivatives, such as carboxymethyl cellulose, hydroxypropyl cellulose, hydroxyethyl cellulose, hydroxypropyl methyl cellulose, hydroxypropyl ethyl cellulose, sodium carboxymethyl cellulose, ethyl or propyl cellulose, polyacrylic acids, polyacrylates, polyvinylpyrrolidones, vinylpyrrolidone-vinyl acetate copolymers, polyvinyl alcohols, polyethylene oxide polymers, polyacrylamides, polyethylene glycols, gelatine, collagen, alginates, pectin, pullulan, tragacanth, chitosan, alginic acid, arabinogalactan, galactomannan, agar, agarose, carrageenan, and/or natural gums, polyvinyl alcohols being particularly preferred.

The oral thin film is preferably characterized in that the at least one polymer, preferably the water-soluble polymer, is present in the matrix layer in an amount of 10 to 100% (w/w), or 10 to 99% (w/w), or 30 to 95% (w/w), preferably 60 to 90% (w/w), particularly preferably 70 to 90% (w/w), based on the total weight of said matrix layer.

The composite according to the invention is preferably characterized in that the oral thin film as a whole is water-soluble. Here too, water-soluble is preferably understood to mean a solubility of more than 100 g/L in water at 25° C.

The composite according to the invention is preferably characterized in that the oral thin film comprises at least one active pharmaceutical ingredient.

The at least one active pharmaceutical ingredient in the oral thin film is preferably selected from the group consisting of hypnotics, sedatives, antieleptics, amphetamines, psychoneurotropics, neuroleptics, neuromuscular-blocking agents, antispasmodics, antihistamines, antiallergics, cardiotonic agents, antiarrhythmics, diuretics, hypotensive agents, vasopressors, antitussives, expectorants, analgesics, thyroid hormones, sex hormones, glucocorticoid hormones, antidiabetics, antitumor agents, antibiotics, chemotherapeutics, narcotics, anti-Parkinson agents, anti-Alzheimer agents, and/or triptans, wherein this group is not exhaustive.

Specific examples comprise acetaminophen, adrenaline, alprazolam, amlodipine, anastrozole, apomorphine, aripiprazole, atorvastatin, baclofen, benzocaine, benzocaine/menthol, benzydamine, buprenorphine, buprenorphine/naloxone, buprenorphine/naloxone/cetirizine, cetirizine, chlorpheniramine, clomipramine, dexamethasone, dextromethorphan, dextromethorphan/phenylephrine, diclofenac, diphenhydramine, diphenhydramine/phenylephrine, donepezil, dronabinol, epinephrine, escitalopram, famotidine, fentanyl, glimepiride, GLP-1 peptides, granisetron, insulin, insulin nanoparticles, insulin/GLP-1 nanoparticles, ketamine, ketoprofen, ketotifen, caffeine, levocetirizine, loperamide, loratadine, meclizine, methylphenidate, midazolam, mirodenafil, montelukast, multimeric-001, naloxone, nicotine, nitroglycerin, olanzapine, olopatadine, ondansetron, oxybutynin, pectin, pectin/menthol, pectin/ascorbic acid, pediaSUNAT (artesunate and amodiaquine), piroxicam, phenylephrine, prednisolone, pseudoephedrine, risperidone, rivastigmine, rizatriptan, selegilines, senna glycosides, sildenafil citrate, simethicone, sumatriptan, tadalafil, testosterone, triamcinolone acetonide, triptan, tropicamide, voglibose, zolmitriptan, zolpidem, and/or pharmaceutically acceptable salts of these compounds.

The active pharmaceutical ingredient may also be a mixture of different active ingredients.

The amount of active ingredient in the oral thin film depends on its type and is usually 0.01 to 70% (w/w), preferably 0.1 to 50% (w/w), particularly preferably 1 to 40% (w/w), based on the total weight of the oral thin film.

It may be advantageous that the oral thin film further contains at least one excipient selected from the group comprising colorants, flavors, sweeteners, taste-masking agents, surfactants, enhancers, pH regulators, preservatives, antioxidants, and/or plasticizers.

These excipients are preferably each contained in an amount of 0.001 to 20% (w/w) in the oral thin film.

In principle, the oral thin film is not limited in the number of layers it can contain.

Thus, embodiments are conceivable in which the oral thin film comprises further layers. The above definitions apply analogously to the other layers.

The composite according to the invention is preferably characterized in that the application aid and the oral thin film are joined to each other by means of an adhesive layer, preferably a water-soluble adhesive layer, by means of a seal, preferably a heat seal, and/or are joined to each other by means of coating one on top of the other.

An adhesive layer is understood to be a layer that can act as an adhesive, as defined in DIN EN 923:2016-03.

Water-soluble adhesive layers are particularly suitable as adhesive layers, as described in DE 10 2014 127 452 A1, the relevant content of which is hereby included in full.

Suitable adhesive layers comprise at least one water-soluble polymer and at least one plasticizer, wherein the at least one water-soluble polymer in the at least one water-soluble adhesive layer comprises shellac, a vinylpyrrolidone-vinyl acetate copolymer, a polyvinylcaprolactam-polyvinyl acetate-polyethylene glycol copolymer, hydroxypropyl cellulose or hydroxypropyl methyl cellulose, and/or polyvinylpyrrolidone, and wherein the at least one plasticizer in the at least one water-soluble adhesive layer comprises glycerol, polyethylene glycol, in particular polyethylene glycol 200, and/or tributyl citrate. The weight ratio of the at least one water-soluble polymer to the at least one plasticizer in the at least one adhesive layer is preferably about 85 to 50 to about 15 to 50.

Such an adhesive layer, which contains at least one water-soluble polymer and at least one plasticizer, can firmly adhere two further layers, which are not sticky themselves, to each other as an intermediate water-soluble adhesive layer, and thus enable the formation of multilayer composites.

Sealing is understood to mean any possible way in which the application aid and the oral thin film can be joined to each other. However, this does not concludingly comprise joints by embossing and/or heat sealing.

Heat sealing refers to the joining of the application aid and the oral thin film by punctiform heating and pressing. The punctiform heating melts the at least one polymer that is present in the application aid or in the oral thin film, which leads to a firm joint after renewed cooling. Preferably, the temperature is punctiformly raised to a temperature that is above the melting temperature or glass transition temperature of the respective polymer.

The usual temperatures for heat sealing are about 50° C. to 200° C.

The heat sealing is preferably carried out for about 5 seconds, preferably for about 3 seconds, and particularly preferably for about 2 seconds or less at a temperature of about 50° C. to 200° C.

Coating one on top of the other is preferably understood to mean the direct application of a second layer on top of an existing first layer.

For example, the application aid can be provided and the composition of the oral thin film is applied (coated) to the application aid in a processable form, for example as a solution, emulsion, or suspension. After drying, a strong composite is thus formed. It is also possible to provide the oral thin film and coat it with the application aid.

The composite according to the invention is further preferably characterized in that the application aid dissolves in the oral cavity of a patient within 20 s to 5 min, preferably from 30 s to 3 min.

The composite according to the invention is further preferably characterized in that the application aid and the oral thin film dissolve in the oral cavity of a patient within 20 s to 5 min, preferably from 30 s to 2 min.

The present invention also relates to a method for preparing the composite, preferably as described above, comprising the steps of:
providing an application aid,
providing an oral thin film, and
firmly joining the application aid to the oral thin film.

All embodiments defined above for the composite, the oral thin film, and the application aid apply analogously to the method according to the invention.

It is irrelevant, here, whether the application aid or the oral thin film is provided in the first step.

Preferably, the application aid and/or the oral thin film is provided by a method that preparing a solution, emulsion, melt, or suspension comprising all ingredients of the application aid or the oral thin film, respectively, followed by spreading and drying the solution, emulsion, melt, or suspension.

All suitable shapes can be cut or punched out of these dried solutions, emulsions, melts, or suspensions.

If microneedles are to be present, they are preferably attached to the oral thin film with an adhesive layer. For example, an adhesive layer such as that defined above for joining the application aid to the oral thin film is suitable here as an adhesive layer.

Alternatively, the oral thin film with microneedles can also be produced by using a negative mold of the microneedles during the production of the oral thin film, so that the oral thin film and the microneedles are monolithic.

Alternatively, the microneedles can be attached to the oral thin film using any suitable means of attachment.

If the application aid and/or the oral thin film is present in the form of a foam, the method also comprises the step of foaming the solution, emulsion, melt, or suspension by introducing a gas or gas mixture, by chemical gas generation, or by expansion of a dissolved gas.

The firm joining is preferably achieved by means of a water-soluble adhesive layer, by means of a seal, preferably a heat seal, and/or by means of coating one on top of the other, as defined above.

The present invention further relates to a composite obtainable according to the method described above.

All embodiments defined above for the composite, the oral thin film, and the application aid apply analogously to a composite obtainable by the method described above.

Additionally, the present invention relates to a composite as described above or obtainable by the method described above as a medicinal product.

All embodiments defined above for the composite, the oral thin film, and the application aid apply analogously to the medical use according to the invention.

### DESCRIPTION OF THE FIGURES

FIG. 1:

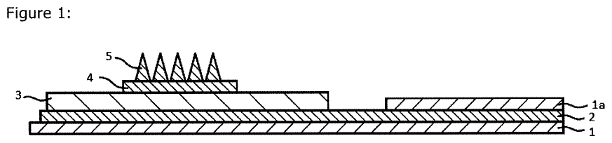

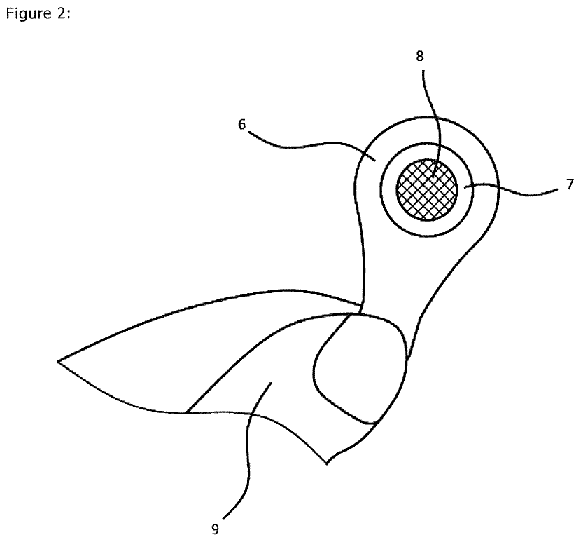

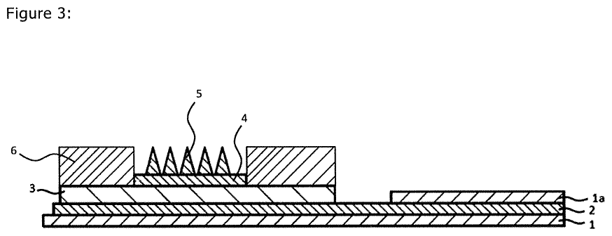

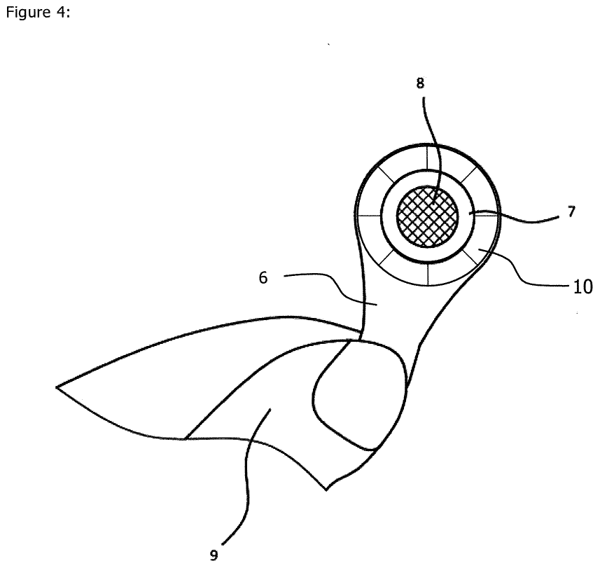

The FIG. 1 shows a possible embodiment of the composite, comprising an oral thin film and an application aid for the oral thin film. The composite has a first area of the application aid on which the oral thin film is disposed, and a second area of the application aid which is designed in such a way that a user can touch the application aid in such a way that the oral thin film is not touched.

An adhesive layer (2) is applied to an application aid (1), to which the oral thin film (3) is applied in a first area. Optionally, a piece of the application aid (1a) can once again be applied to the adhesive layer in a second area, so that the user does not have to touch the adhesive layer.

The oral thin film may optionally be equipped with microneedles (5). These microneedles (5) can either be applied to the oral thin film with another adhesive layer (4) or attached thereon using alternative attachment measures.

FIG. 2:

The FIG. 2 shows another possible embodiment of the composite, comprising an oral thin film and an application aid for the oral thin film. Here, the composite has a first area of the application aid on which the oral thin film is disposed, and a second area of the application aid which is designed in such a way that a user can touch the application aid in such a way that the oral thin film is not touched. The user is represented by the stylized hand (9).

An oral thin film (7) is applied to an application aid (6) in an area that differs from the area that the user touches with his hand (9). The oral thin film (7) may optionally be equipped with microneedles (8).

FIG. 3:

The FIG. 3 shows a cross section of a possible embodiment of the composite, comprising an oral thin film and an application aid for the oral thin film. The composite has a first area of the application aid on which the oral thin film, the microneedles, and a raised area framing the microneedles are disposed to ensure that the microneedles are not damaged.

An adhesive layer (2) is applied to an application aid (1), to which the oral thin film (3) is applied in a first area. Optionally, a piece of the application aid (1a) can once again be applied to the adhesive layer in a second area, so that the user does not have to touch the adhesive layer.

The oral thin film is equipped with microneedles (5), wherein a raised area (6) is attached to the application aid (1), which frames the microneedles (5). The microneedles (5) and the raised area (6) can either be applied to the oral thin film with another adhesive layer (4) or attached there using alternative attachment measures.

FIG. 4:

The FIG. 4 shows another possible embodiment of the composite, comprising an oral thin film and an application aid for the oral thin film. The composite has a first area of the application aid on which the oral thin film, the microneedles, and a raised area framing the microneedles are disposed to ensure that the microneedles are not damaged. The second area of the application aid is designed in such a way that a user can touch the application aid in such a way that the oral thin film is not touched. The user is represented by the stylized hand (9).

An oral thin film (7) is applied to an application aid (6) in an area that differs from the area that the user touches with his hand (9). The oral thin film (7) is equipped with microneedles (8), wherein a raised area (10) is attached to the application aid (6), which frames the microneedles (8).

The invention is explained below in more detail with reference to non-limiting examples.

### EXAMPLES

An oral thin film, in this case a multilayer system with microneedles, was provided with a polyvinyl alcohol foam as an application aid.

The oral thin film was applied to the application aid using an adhesive layer.

The application aid has the following formulation.

| Material | Content %(dry) | Description |
|---|---|---|
| polyvinyl alcohol 4-88 | 84.8 | polymer |
| FD&C-Red no. 40 | 0.2 | dye |
| saccharin Na | 1.0 | sweetener |
| sucralose | 2.0 | sweetener |
| glycerol | 5.0 | plasticizer |
| cherry flavor | 6.0 | flavoring agent |
| menthol | 1.0 | flavoring agent |

A stable composite is obtained. The very thin oral thin film, which tends to crease, could be applied. The presence of the microneedles in particular makes appropriate application necessary, since an inappropriate application will lead to the microneedles breaking off.

1. A composite comprising an oral thin film and an application aid for the oral thin film, characterized in that the oral thin film and the application aid are firmly joined to each other.

2. The composite according to claim 1, characterized in that the application aid comprises a first area and a second area, wherein the oral thin film is disposed in the first area of the application aid, and the second area of the application aid is designed such that a user can touch the application aid in such a way that the oral thin film is not touched.

3. The composite according to claim 1, characterized in that the application aid has a surface area which is at least twice as large as the surface area of the oral thin film.

4. The composite according to claim 1, characterized in that the application aid has a surface area of 1 cm² to 30 cm².

5. The composite according to claim 1, characterized in that the application aid comprises at least one water-soluble polymer.

6. The composite according to claim 1, characterized in that the application aid as a whole is water-soluble.

7. The composite according to claim 1, characterized in that the application aid comprises flavors, flavor masking agents and/or anesthetics.

8. The composite according to claim 1, characterized in that the oral thin film comprises an oral thin film in the form of a film, in the form of a foam, and/or in the form of a microneedle system.

9. The composite according to claim 1, characterized in that the oral thin film comprises at least one water-soluble polymer.

10. The composite according to claim 1, characterized in that the oral thin film comprises at least one active pharmaceutical ingredient.

11. The composite according to claim 1, characterized in that the application aid and the oral thin film are joined to each other by means of an adhesive layer by means of a seal and/or are joined to each other by means of coating one on top of the other.

12. The composite according to claim 1, characterized in that the application aid dissolves in the oral cavity of a patient within 20 s to 5 min.

13. A method for preparing a composite according to claim 1, comprising the steps of:
providing an application aid,
providing an oral thin film, and
firmly joining the application aid to the oral thin film.

14. The method according to claim 13, characterized in that the firm joining is carried out by means of an adhesive layer, by means of a seal and/or by means of coating one on top of the other.

15. A method for the administration of a pharmaceutical ingredient comprising providing a composite according to claim 1 to a patient.

16. The composite according to claim 1, characterized in that the application aid has a surface area 2 to 10 times as large as the surface area of the oral thin film.

17. The composite according to claim 1, characterized in that the application aid and the oral thin film are joined to each other by means of a water-soluble adhesive layer.

18. The composite according to claim 1, characterized in that the application aid and the oral thin film are joined to each other by means of a heat seal.

19. The method according to claim 13, characterized in that the firm joining is carried out by means of a water-soluble adhesive layer and/or by means of a heat seal.
